(19) United States
(12) Patent Application Publication
(10) Pub. No.: US 2021/0221693 A1
(43) Pub. Date: Jul. 22, 2021

(54) CARBON DIOXIDE CAPTURING STEAM METHANE REFORMER

(71) Applicant: FuelCell Energy, Inc., Danbury, CT (US)

(72) Inventor: Fred C. JAHNKE, Rye, NY (US)

(21) Appl. No.: 16/097,162

(22) PCT Filed: Apr. 28, 2017
(86) PCT No.: PCT/US2017/030230

§ 371 Date: Oct. 26, 2018

Related U.S. Application Data
(60) Provisional application No. 62/329,707, filed on Apr. 29, 2016.

(51) Int. Cl. C01B 32/50; C01B 3/48; C07C 1/12; C07C 1/04; H01M 8/14; H01M 8/0612; H01M 8/04007; H01M 8/04701; H01M 8/0656; C25B 1/02; C25B 9/09

(52) U.S. Cl. CPC C01B 32/50; H01M 2008/147; C07C 1/12; C07C 1/0485; H01M 8/14; H01M 8/0618; H01M 8/04007; H01M 8/04716; H01M 8/0656; C25B 1/02; C25B 9/09; C01B 2203/0222; C01B 2203/0216; C01B 2203/067; C01B 2203/1241; C01B 2203/148; C01B 2203/041; C01B 2203/0475; C01B 3/48

(57) ABSTRACT

An integrated system for carbon dioxide capture includes a steam methane reformer and a CO₂ pump that comprises an anode and a cathode. The cathode is configured to output a first exhaust stream including oxygen and carbon dioxide and the anode is configured to receive a reformed gas from the steam methane reformer and to output a second exhaust stream that includes greater than 95% hydrogen.

### RELATED APPLICATIONS

The present application claims priority to U.S. Provisional Application No. 62/329,707, filed on Apr. 29, 2016, which is hereby incorporated by reference in its entirety.

### BACKGROUND

The present disclosure relates to a Steam Methane Reformer (SMR). In particular, the present disclosure relates to a SMR with enhanced CO₂ capture.

Steam methane reformers (SMRs) are generally used to produce a syngas from a gas feedstock such as natural gas or refinery gas. The produced syngas can be further processed within the plant to yield various end products, including purified hydrogen, methanol, carbon monoxide and ammonia. However, the flue gas produced during the reforming process contains many contaminants, such as carbon dioxide. These contaminants are known to adversely affect the environment by contributing to overall climate change. SMR's are known to be one of the largest carbon dioxide (CO₂) emitters in refinery systems. As such, in recent years, many government regulatory bodies have required the reduction in emissions of these contaminants, in particular carbon dioxide, into the atmosphere.

Given the recognition of the harmful effect of carbon dioxide release and recent restrictions on its emission, efforts have been made to efficiently remove carbon dioxide in a purified form from a flue gas produced by a reformer plant. By removing carbon dioxide from the flue gas, the carbon dioxide alternatively may be used for other, safer purposes, such as underground storage or oil production needs.

Current methods for CO₂ capture from flue gas, such as for example, using an amine absorption stripper system or a molten carbonate fuel cell (MCFC) fuel cell running in fuel cell mode, are highly inefficient. This is due, in part, to the dilute concentration of carbon dioxide present in the flue gas, which can be as little as 5% in concentration. The amine systems are generally too energy intensive, and the MCFC fuel cell incurs a substantial voltage penalty due to the dilution of the cathode with the large quantity of nitrogen contained in flue gas lowering the efficiency and output of the fuel cell. As such, conventional systems designed to remove CO₂ can be very costly and require a high input of energy to sufficiently remove or reduce the CO₂, significantly reducing the production capabilities of the refinery itself.

### SUMMARY

Embodiments described herein provide a SMR-CO₂ capture system that generates pure CO₂, as well as pure H₂, such that a higher output value may be realized, further offsetting the costs of capturing CO₂ and increasing the overall efficiency of the power plant. The system also has zero NOx emissions, since combustion is done without the presence of N2.

In certain embodiments, a SMR-CO₂ capture system includes a CO₂ pump referred to as a Reforming-Electrolyzer-Purifier (REP) in a related patent application WO2015/116964 configured to receive a reformed gas from a SMR and output a first exhaust stream comprising oxygen and carbon dioxide and a second exhaust stream containing a high concentration of hydrogen which can be exported as a valuable by-product.

In certain embodiments, an integrated system for carbon dioxide capture is provided, which includes a steam methane reformer; and a CO₂ pump comprising an anode and a cathode; wherein the cathode is configured to output a first exhaust stream and the anode is configured to receive a reformed gas from the steam methane reformer and to output a second exhaust stream; wherein the first exhaust stream comprises oxygen and carbon dioxide; and wherein the second exhaust stream comprises greater than 95% hydrogen.

In certain embodiments, natural gas and steam are fed to a reformer and the outlet reformed gas from the reformer is fed to a high temperature CO₂ pump (REP). In certain embodiments, the CO2 pump (REP) is a MCFC fuel cell running in reverse. In certain embodiments, the CO2 pump (REP) converts the residual methane (CH₄) to hydrogen (H₂) and the carbon monoxide (CO) to CO₂. The CO₂/oxygen mixture generated by the CO₂ pump (REP) can be recycled back to the reformer to be used in place of air, and essentially all of the methane and hydrogen used as fuel to the reformer is converted into CO₂ and water. In certain embodiments, the flue gas from the reformer is essentially pure CO₂ which can be cooled and almost all water removed by condensation.

In certain embodiments, the CO₂ pump (REP) anode effluent is cooled and transported to a methanator, where the residual CO and CO₂ are converted back into methane. Without CO in the gas, the methanator outlet can be fed to Electrochemical Hydrogen Compressor (EHC) to generate pure H₂ at pressure and an off-gas stream with the residual methane and residual H₂. The off-gas stream generated from the EHC may be recycled as fuel to the SMR or recycled as feed to the SMR. If recycled as feed to the SMR, another fuel, such as methane, will be needed in the SMR to heat balance the system. The hydrogen generated in the CO₂ capture system could be used in a low-temperature fuel cell to load follow and produce peak power, or could be exported for fuel-cell vehicles and other industrial uses. The hydrogen could be used in a low temperature fuel cell after methanation, but before purification if desired.

In certain embodiments, an integrated SMR-carbon dioxide capture system removes carbon dioxide from a reformer system. The carbon dioxide is delivered to the CO₂ pump (REP) which generates an output of a first exhaust stream comprising oxygen and carbon dioxide and a second exhaust stream containing a high concentration of hydrogen from water.

In one aspect, which is combinable with the above embodiments and aspects, the CO₂ pump (REP) is a molten carbonate fuel cell operating in reverse, and configured to receive reformed gas from a reformer to produce CO₂. In another aspect, the CO₂ gas source for the CO2 pump (REP) is a steam methane reformer.

In one aspect, which is combinable with the above embodiments and aspects, the SMR is operated at lower than typical temperature and higher than typical steam feed, allowing lower cost materials to be used in the SMR. Completion of the reforming reaction then occurs in the REP

In one aspect, which is combinable with the above embodiments and aspects, the CO₂ pump (REP) is configured to produce a first exhaust stream comprising mainly CO₂ and oxygen. The CO₂ pump (REP) is also configured to produce a second exhaust stream comprising mainly hydrogen. In one aspect, which is combinable with the above embodiments and aspects, the first exhaust stream comprises greater than about 95% of the feed carbon dioxide.

In certain embodiments, a carbon dioxide capture system for removing carbon dioxide from a reformer includes a CO₂ pump (REP) having an anode and a cathode. The anode is configured to receive a reformed natural gas and output an enriched hydrogen stream. The cathode is configured to output a mixture of CO₂ and O₂ in approximately a 2/1 ratio. In one aspect, the CO₂ pump (REP) is a molten carbonate fuel cell operating in reverse as an electrolyzer.

In one aspect, which is combinable with the above embodiments and aspects, the hydrogen enriched anode exhaust stream is partially cooled and transported to a methanator that is configured to convert the residual CO and CO₂ to methane. In another aspect, the third exhaust stream from the methanator is transported to a electrochemical hydrogen compressor that is configured to receive the exhaust stream. The third exhaust stream may include hydrogen and methane and CO₂, but essentially no CO.

In certain embodiments, capturing carbon dioxide from a reformed gas is provided, which includes supplying a reformed gas to CO₂ pump; and outputting, from the CO₂ pump, a first exhaust stream comprising carbon dioxide and oxygen and a second exhaust stream comprising hydrogen.

In one aspect, which is combinable with the above embodiment, the method for capturing the carbon dioxide further includes transporting the CO₂ and oxygen back to the reformer to convert the methane and hydrogen used as fuel to the reformer into CO₂ and water.

In one aspect, which is combinable with the above embodiment, the method for capturing the carbon dioxide further includes sequestering substantially all of the carbon dioxide from the reformer flue gas.

In one aspect, which is combinable with the above embodiments and aspects, the method for capturing the carbon dioxide further includes supplying a natural gas and water to the reformer.

In one aspect, which is combinable with the above embodiments and aspects, the method for capturing the carbon dioxide further includes transporting a second exhaust stream comprising hydrogen with small amounts of CO, CO₂ and CH₄ to a methanator.

In one aspect, which is combinable with the above embodiments and aspects, the method for capturing the carbon dioxide further includes optionally cooling the second exhaust stream comprising mainly hydrogen prior to transporting it to a methanator to generate a third exhaust stream.

In one aspect, which is combinable with the above embodiments and aspects, the method includes transporting the third exhaust stream generated in the methanator to an electrochemical hydrogen compressor.

In one aspect, which is combinable with the above embodiments and aspects, the method further includes separating hydrogen from the residual methane in an electrochemical hydrogen compressor to produce a purified hydrogen stream.

In one aspect, which is combinable with the above embodiments and aspects, the method further includes separating hydrogen from the residual methane in an electrochemical hydrogen compressor and increasing the pressure of the purified hydrogen.

In one aspect, which is combinable with the above embodiments and aspects, the method for capturing the carbon dioxide further includes outputting a pure hydrogen gas stream from the electrochemical hydrogen compressor. In certain embodiments, the pure hydrogen gas includes greater than 98% hydrogen, typically greater than 99.9% hydrogen.

The foregoing is a summary of the disclosure and thus by necessity contains simplifications, generalizations, and omissions of detail. Consequently, those skilled in the art will appreciate that the summary is illustrative only and is not intended to be in any way limiting. Other aspects, features, and advantages of the devices and/or processes described herein, as defined by the claims, will become apparent in the detailed description set forth herein and taken in conjunction with the accompanying drawings.

### BRIEF DESCRIPTION OF THE DRAWINGS

FIG. 1 shows a schematic view of a standard SMR configuration.

FIG. 2 shows a schematic view of a SMR system utilizing a CO₂ pump (REP), in accordance with a representative embodiment.

FIG. 3 shows a detail schematic view of the SMR-CO₂ capture system of FIG. 2 in accordance with a representative embodiment.

FIG. 4 shows a schematic view of the SMR-CO₂ capture system, according to an exemplary embodiment.

### DETAILED DESCRIPTION

Referring generally to the figures, disclosed herein is an enhanced SMR-CO₂ capture system capable of producing a highly purified CO₂ flue gas while co-producing a highly pure hydrogen syngas for additional energy needs that is both less costly and highly efficient in terms of energy production.

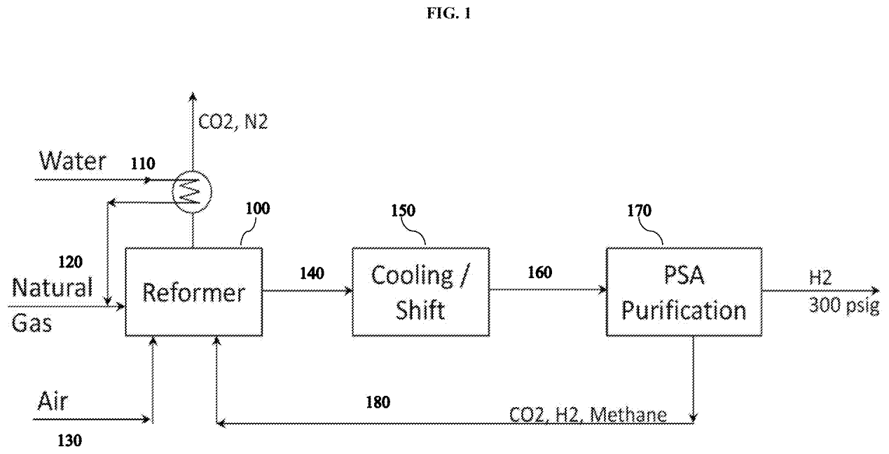

FIG. 1 shows a typical standard SMR configuration. Steam supplied by a steam supply line 110 and natural gas supplied by a natural gas supply line 120 and are mixed and fed to a reformer 100 for converting the methane to hydrogen CO₂, and CO. The reformer effluent is transported through effluent line 140 to a shifting assembly 150, where the effluent is cooled and most of the CO is shifted to hydrogen. The shifted gas is then sent via shift gas line 160 to a PSA (pressure swing adsorption) system 170 where the hydrogen is separated from the residual methane and CO in the gas along with the CO₂ produced from the reforming and shift reactions. The residual gases are recycled as fuel to the reformer 100 via the recycling line 180, where the gases are combusted with air supplied by an air supply line 130 to provide the heat needed for the endothermic reforming reaction. All of the CO₂ generated in the production of the hydrogen is vented in the reformer flue gas as a mixture of N₂, CO₂, and H₂O with some NOx. Typically the SMR is the largest CO₂ emitter in a refinery.

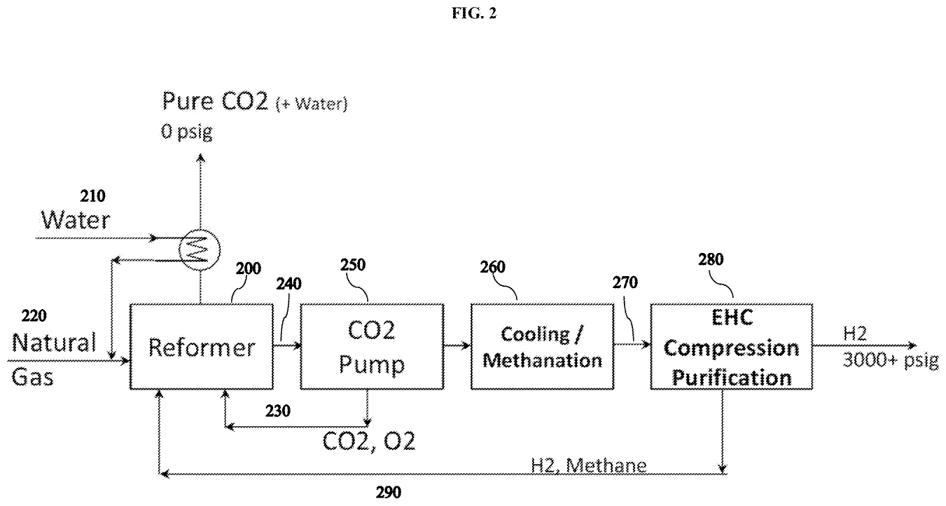

FIG. 2 shows a SMR-CO₂ capturing system which includes a CO₂ pump (REP) 200 for capturing carbon dioxide and producing hydrogen. As shown in FIG. 2, steam supplied by a steam supply line 210 and natural gas supplied by a natural gas supply line 220 are mixed and fed to a reformer 200 for converting the methane to hydrogen and CO. This reformer operates at a lower temperature than a standard reformer since residual methane in the reformed gas is also converted to H₂ in the CO₂ pump (REP). This reduces the cost of the reformer substantially. The reformer effluent is transported through effluent line 240 and introduced in to a high temperature CO₂ pump (REP) 250. In the embodiment shown in the Figures, the CO₂ pump (REP) 250 comprises a molten carbonate fuel cell (MCFC) operating in reverse in electrolysis mode. In some embodiments, the CO₂ pump (REP) 250 may further comprise a plurality of individual cells connected to form a fuel cell stack. During operation of the pump 250 as a reverse MCFC unit, the residual methane is converted to hydrogen and CO is converted to CO₂ and pumped across the membrane as a CO₃⁻ ion where the third oxygen comes from the separation of water into H₂ and O⁻. Since the CO₂ is removed electrochemically at a high temperature, the equilibrium of the reforming reaction can be pushed close to completion.

In the pump, CO₂ reacts with water to create CO₃⁻ according to the following reaction:

CO₂+H₂O↔CO₃^{═}⬆+H₂

This reaction is driven forward by the electrochemical removal of the CO₃⁻ ion so that near pure {˜98%) hydrogen is generated. The MCFC unit which is used as the CO₂ pump (REP) generates a cathode exhaust stream and an anode exhaust stream. The cathode exhaust stream, which substantially contains oxygen and carbon dioxide, is removed from the CO₂ pump (REP) 250 and recycled through a cathode exhaust line 230 to the reformer system 200. At this point, the cathode exhaust stream may include about 66% of carbon dioxide and 34% O₂. This stream can be used in place of air normally used in the combustor of the SMR. The absence of N₂ in the stream means that the flue gas from the SMR is now only CO₂ and water with traces of unreacted O₂. If desired, the trace O₂ can be minimized by catalytically reacting the O₂ with a stoichiometric amount of fuel or H₂. Thus CO₂ and O₂ recycled to the reformer produce a pure CO₂ exhaust gas once the gas is cooled and the water condensed from the flue gas which is removed from the reformer along with water. The gas is then further cooled and compressed so that the CO₂ is captured. Carbon dioxide is then removed from the reformer system where the CO₂ may be stored for other purposes.

As further shown in FIG. 2, the effluent from the CO₂ pump (REP), which is over 95% hydrogen, is cooled and passed through a methanator 260 where the residual CO and much of the CO₂ are converted back into methane so that CO does not impact downstream processes. The effluent from the methanator (third exhaust stream) is transferred through an exhaust line 270 to a electrochemical hydrogen compressor (EHC) 280, where the hydrogen gas is purified and compressed. This allows the hydrogen to be stored at pressure and/or exported. Additional hydrogen is generated from the electrolysis reaction and added to the hydrogen from reforming methane. The value of the additional hydrogen generated offsets most or all of the cost of the power needed by the pump.

Alternately, mechanical compression and a small PSA could be used to increase the pressure of the H₂ and purify the H₂ (not shown). Since a PSA is not poisoned by CO, methanation is not required if a PSA is used.

The pure hydrogen gas generated using the present systems and methods may include greater than about 95% hydrogen. The pure hydrogen generated may include greater than about 96%, greater than about 86.5%, greater than about 97%, greater than about 97.5%, greater than about 98%, greater than about 98.5%, or greater than about 99% hydrogen. In an exemplary embodiment, the pure hydrogen gas includes greater than 98% hydrogen. In an exemplary embodiment, the pure hydrogen prior to purification (e.g., prior to feeding to EHC) may include greater than about 95% hydrogen, and after purification (e.g., output from EHC) may include greater than about 99.9% hydrogen.

The generated hydrogen could be used in a low-temperature fuel cell to load follow and produce peak power or it could be exported for use in fuel-cell vehicles or other industrial uses. The EHC not only removes the residual methane but also increases the pressure of the hydrogen. The exhaust stream from the EHS, comprising mainly of methane and hydrogen exits the EHS through a recycle line 290 where the exhaust stream is recycled back to the reformer 200. This recycled exhaust may be used as fuel for the reform or feed to the reformer. A blower may be needed to recycle the exhaust gas as feed to the reformer.

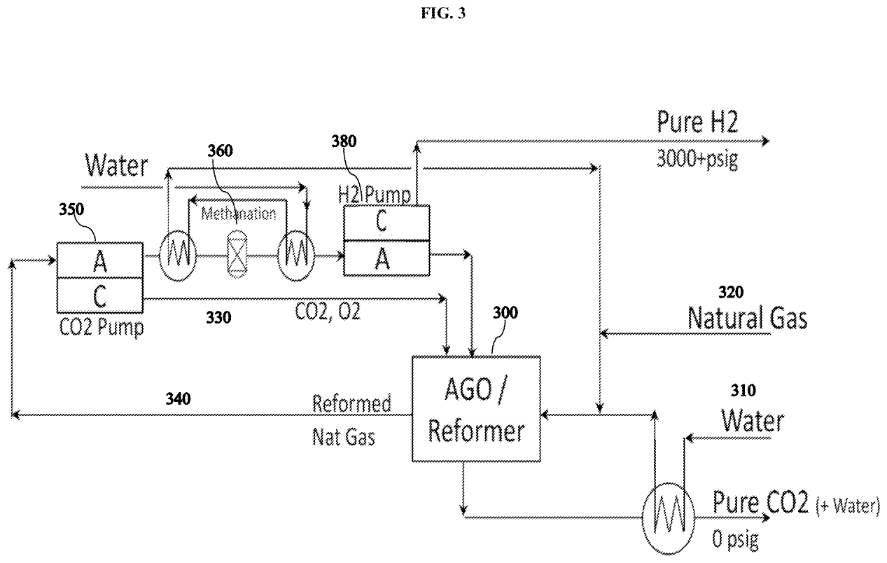

FIG. 3 is a detailed, close-up view of the embodiment of the SMR-CO₂ capture system depicted in FIG. 2. As shown in FIG. 3, once the cathode exhaust stream comprising CO₂ and O₂ is transported back through a cathode exhaust line 330 to the reformer 300, the anode exhaust stream comprised mainly of hydrogen is cooled and then sent to a methanator 360. In the methanator, all of the residual CO and most of CO₂ are converted back into methane. Removal of all CO in the gas helps to minimize the power requirement of the electrochemical hydrogen compressor. The CO₂ pump (REP) 350 generates a mixture of two thirds carbon dioxide and one third oxygen by transferring electrochemically the CO₃⁻ ion across the high temperature membrane. This CO₂ oxygen mixture can be used in place of air in the reformer. By replacing the air with CO₂ and oxygen, essentially all of the methane and hydrogen used as fuel in the reformer are converted into CO₂ and water. Thus the flue gas output from the reformer is essentially pure CO₂ after it is cooled and the water is condensed out. All of the CO₂ from the system can be sequestered by compressing this gas without the need for further purification. Since no nitrogen is present, there is the additional advantage that no in NOx is produced or emitted.

The SMR-CO₂ capture system has several advantages over standard SMR, such as:
CO₂ is produced which is ready for capturing.
No NOx emissions even if exhaust vented.
The purified hydrogen produced is at pressure, preferably 3000 psig or greater.
High conversion of the methane to hydrogen means that the system remains in heat balance with no excess heat that must be converted to steam or other byproducts.
The system is scalable from a small home 1 kg/day system to 2,000+kg/day.
The equipment used in the system is the same as currently used for MCFC fuel cells and thus is readily available.
About 20% of the hydrogen produced is from the water-CO₂ reaction, reducing the fuel consumption of the system.
The system can be operated to load follow, if needed, to meet the hydrogen demand. It could also be used to load follow to help balance the power requirements of the area.

The cost of the power required to operate the CO₂ pump (REP) and the electrochemical hydrogen separator is offset by the hydrogen produced from water which is extremely efficient at the high temperature of the CO₂ pump (REP). Further, the high hydrogen pressure should eliminate or reduce downstream compression power.

### Example 1

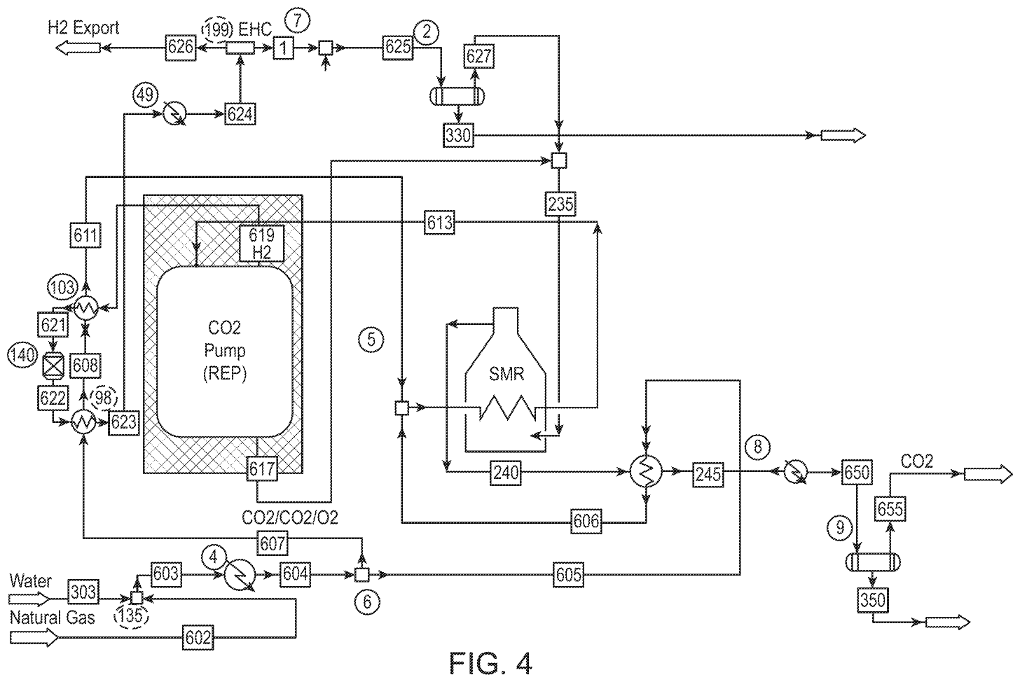

A detailed heat and material balance was performed on the SMR-CO₂ capture system based on a 30 cell DFC stack. This system would be expected to produce 122 kg/day of H₂ at 3000 psig with no moving parts. Raw H₂ production efficiency (excluding compression power) is 70 to 93% depending on the how the power is included in the calculations and the voltage assumptions of the CO₂ pump (REP) and the EHC. The efficiency of the pure, 3000 psig H₂ is still 76% (excluding power production efficiency). If steam is used for the water source, the system is in heat balance when heat losses are included. If liquid water is used for the water source, an additional 3-5% of energy is needed. Steam based heat and material balance (HMB) balance is shown in FIG. 4 and Table 1.

TABLE 1
CO₂ Pump w Stm Ref 8-8-13b.xlsm

| Stream No. | 602 | 303 | 621 | 622 | 626 |
|---|---|---|---|---|---|
| Name | Natural Gas | Water/Steam | H2 to | H2 from | H2 Export |
| | Feed | Feed | Methanator | Methanator | |
| Molar flow | 1.40 | 4.00 | 6.69 | 6.68 | 5.60 |
| lbmol/hr | | | | | |
| Mass flow | 22.5 | 72.1 | 18.8 | 18.8 | 11.3 |
| lb/hr | | | | | |
| Temp F. | 100° | 255° | 500° | 501° | 176° |
| Pres psia | 20.00 | 30.00 | 19.98 | 19.98 | 3,000.00 |

| | lb- | mole | lb- | mole | lb- | mole | lb- | mole | lb- | mole |
|---|---|---|---|---|---|---|---|---|---|---|
| Components | mole/hr | % | mole/hr | % | mole/hr | % | mole/hr | % | mole/hr | % |
| Hydrogen | 0.00 | 0.00 | 0.00 | 0.00 | 6.34 | 94.83 | 6.34 | 94.82 | 5.60 | 100.00 |
| Methane | 1.40 | 100.00 | 0.00 | 0.00 | 0.11 | 1.72 | 0.12 | 1.73 | 0.00 | 0.00 |
| Carbon | 0.00 | 0.00 | 0.00 | 0.00 | 0.00 | 0.01 | 0.00 | 0.00 | 0.00 | 0.00 |
| Monoxide | | | | | | | | | | |
| Carbon | 0.00 | 0.00 | 0.00 | 0.00 | 0.00 | 0.00 | 0.00 | 0.00 | 0.00 | 0.00 |
| Dioxide | | | | | | | | | | |
| Water | 0.00 | 0.00 | 4.00 | 100.00 | 0.23 | 3.44 | 0.23 | 3.45 | 0.00 | 0.00 |
| Nitrogen | 0.00 | 0.00 | 0.00 | 0.00 | 0.00 | 0.00 | 0.00 | 0.00 | 0.00 | 0.00 |
| Oxygen | 0.00 | 0.00 | 0.00 | 0.00 | 0.00 | 0.00 | 0.00 | 0.00 | 0.00 | 0.00 |
| Total | 1.40 | 100.00 | 4.00 | 100.00 | 6.69 | 100.00 | 6.68 | 100.00 | 5.60 | 100.00 |

| | | |
|---|---|---|
| ppm CO | 50 ppm | <1 ppm |

| Stream No. | 627 | 330 | 617 | 655 | 350 |
|---|---|---|---|---|---|
| Name | EHC Exh Gas to | EHC Condensate | CO2/O2 from | Cooled CO2 for | Cooled CO2 |
| | Reformer as fuel | from Exh Gas | CO2 Pump | Export/Capture | Condensate |

| | | | |
|---|---|---|---|
| 0.01 | gpm | 0.04 | gpm |

| | | | | | |
|---|---|---|---|---|---|
| Molar flow | 0.90 | 0.18 | 1.88 | 1.42 | 0.99 |
| lbmol/hr | | | | | |
| Mass flow | 4.2 | 3.3 | 75.7 | 62.0 | 17.9 |
| lb/hr | | | | | |
| Temp F. | 101° | 101° | 1100° | 95° | 95° |
| Pres psia | 19.00 | 19.00 | 19.98 | 50.00 | 50.00 |

| | lb- | mole | lb- | mole | lb- | mole | lb- | mole | lb- | mole |
|---|---|---|---|---|---|---|---|---|---|---|
| Components | mole/hr | % | mole/hr | % | mole/hr | % | mole/hr | % | mole/hr | % |
| Hydrogen | 0.74 | 81.98 | 0.00 | 0.00 | 0.00 | 0.00 | 0.00 | 0.00 | 0.00 | 0.00 |
| Methane | 0.12 | 12.80 | 0.00 | 0.00 | 0.00 | 0.00 | 0.00 | 0.00 | 0.00 | 0.00 |
| Carbon | 0.00 | 0.00 | 0.00 | 0.00 | 0.00 | 0.00 | 0.00 | 0.00 | 0.00 | 0.00 |
| Monoxide | | | | | | | | | | |
| Carbon | 0.00 | 0.00 | 0.00 | 0.00 | 1.28 | 68.16 | 1.40 | 98.37 | 0.00 | 0.03 |
| Dioxide | | | | | | | | | | |
| Water | 0.05 | 5.23 | 0.18 | 100.00 | 0.00 | 0.00 | 0.02 | 1.63 | 0.99 | 99.97 |
| Nitrogen | 0.00 | 0.00 | 0.00 | 0.00 | 0.00 | 0.00 | 0.00 | 0.00 | 0.00 | 0.00 |
| Oxygen | 0.00 | 0.00 | 0.00 | 0.00 | 0.60 | 31.84 | 0.00 | 0.00 | 0.00 | 0.00 |
| Total | 0.90 | 100.00 | 0.18 | 100.00 | 1.88 | 100.00 | 1.42 | 100.00 | 0.99 | 100.00 |

The SMR-CO₂ capture system is modular in nature and may be sized for a given location. For example, a plurality of CO2 pump (REP) assemblies may be incorporated into the CO₂ capture system depending on need. Moreover, when based on renewable feedstock, the CO₂ capture system may be capable of producing a highly pure hydrogen gas or hydrogen containing feedstock with negative CO₂ emissions. The result is a system that may realize a lower operating and capital cost, while producing a highly pure CO₂ gas and hydrogen syngas for increased value.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the Figures. It should be noted that the orientation of various elements may differ according to other exemplary embodiments, and that such variations are intended to be encompassed by the present disclosure.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present invention. For example, the heat recovery heat exchangers may be further optimized.

1. An integrated system for carbon dioxide capture comprising:
a steam methane reformer; and
a CO₂ pump comprising an anode and a cathode;
wherein the cathode is configured to output a first exhaust stream and the anode is configured to receive a reformed gas from the steam methane reformer and to output a second exhaust stream;
wherein the first exhaust stream comprises oxygen and carbon dioxide; and
wherein the second exhaust stream comprises greater than 95% hydrogen.

2. The integrated system of claim 1, wherein the CO₂ pump comprises a reforming-electrolyzer-purifier system.

3. The integrated system of claim 1, wherein the reforming-electrolyzer-purifier system comprises a molten carbonate fuel cell running in reverse.

4. The integrated system of claim 1, wherein the reformed gas comprises a natural gas, hydrogen, carbon dioxide, carbon monoxide and water.

5. (canceled)

6. The integrated system of claim 1, wherein the CO₂ pump is configured to convert the residual methane from the steam methane reformer to hydrogen and to convert the carbon monoxide to hydrogen and carbon dioxide.

7. The integrated system of claim 1, wherein the first exhaust stream comprises greater than about 95% of the feed carbon dioxide.

8. The integrated system of claim 1, wherein the cathode is configured to output a mixture of carbon dioxide and oxygen in a ratio of between approximately 1:1 and 4:1.

9. (canceled)

10. The integrated system of claim 8, wherein the system further includes a mechanism for transporting the carbon dioxide and oxygen back to the reformer.

11. The integrated system of claim 1, wherein the second exhaust stream further comprises residual carbon monoxide and carbon dioxide.

12. The integrated system of claim 11, further comprising a methanator that is configured to convert the residual carbon monoxide and a portion of the carbon dioxide from the second exhaust stream to a third exhaust stream comprising methane, hydrogen, and carbon dioxide.

13. The integrated system of claim 12, further comprising an electrochemical hydrogen compressor that is configured to receive the third exhaust stream from the methanator.

14. The integrated system of claim 13, wherein the electrochemical hydrogen compressor is configured to generate pure hydrogen at pressure and an off-gas stream with the residual methane and residual hydrogen.

15. The integrated system of claim 14, wherein the system is configured to recycle the off-gas stream to the steam methane reformer.

16. The integrated system of claim 12, further comprising a low temperature fuel cell that is configured to receive the third exhaust stream from the methanator and generate power.

17. A method for capturing carbon dioxide from a reformed gas comprising:
supplying a reformed gas to CO₂ pump;
outputting, from the CO₂ pump, a first exhaust stream comprising carbon dioxide and oxygen and a second exhaust stream comprising hydrogen; and
transporting the carbon dioxide and oxygen back to the reformer to convert the reformer fuel comprising methane and hydrogen to reformer flue gas comprising carbon dioxide and water.

18. (canceled)

19. The method of claim 17, further comprising sequestering substantially all of the carbon dioxide from the reformer flue gas.

20. (canceled)

21. The method of claim 17, further comprising:
optionally cooling the second exhaust stream,
transporting the cooled second exhaust stream comprising mainly hydrogen to a methanator to generate a third exhaust stream, and
transporting the third exhaust stream from the methanator to an electrochemical hydrogen compressor.

22. (canceled)

23. The method of claim 21, further comprising transporting the third exhaust stream from the methanator to an electrochemical hydrogen compressor, separating hydrogen from the residual methane in the electrochemical hydrogen compressor to produce a purified hydrogen stream and increasing the pressure of the purified hydrocarbon.

24. (canceled)

25. The method of claim 23, further comprising increasing the pressure of the purified hydrocarbon and outputting a pure hydrogen gas stream from the electrochemical hydrogen compressor.

26. The method of claim 25, wherein the pure hydrogen gas comprises greater than 98% hydrogen.
